(19) United States
(12) Patent Application Publication
(10) Pub. No.: US 2026/0272398 A1
(43) Pub. Date: Sep. 17, 2026

(54) ELECTRONIC STETHOSCOPE

(71) Applicant: Murata Manufacturing Co., Ltd., Nagaokakyo-shi (JP)

(72) Inventor: Hirofumi TSUCHIMOTO, Nagaokakyo-shi (JP)

(21) Appl. No.: 19/560,225

(22) Filed: Mar. 9, 2026

(30) Foreign Application Priority Data
Mar. 12, 2025 (JP) 2025-039260

(51) Int. Cl. A61B 7/04

(52) U.S. Cl. CPC A61B 7/04

(57) ABSTRACT

An electronic stethoscope according to the present disclosure is an electronic stethoscope that captures biometric sound. The electronic stethoscope includes a sensor element that captures biometric sound, and a first housing that accommodates the sensor element, that has a pipe shape having a first end and a second end, and that receives a shielding member at the first end. The first housing includes a first flow path that extends through the first housing and that connects a side of an inner wall and a side of an outer wall of the first housing to each other. Around the sensor element, a NULL area in which sensitivity of the sensor element is reduced by greater than or equal to a predetermined value with respect to a maximum gain at which the sensitivity of the sensor element is maximum is defined.

### CROSS REFERENCE TO RELATED APPLICATION

This application claims priority from Japanese Patent Application No. 2025-039260, filed on Mar. 12, 2025. The content of these applications are incorporated herein by reference in its entirety.

### BACKGROUND OF THE DISCLOSURE

### Field of the Disclosure

The present disclosure relates to an electronic stethoscope.

### Description of the Related Art

A known electronic stethoscope captures biometric sound with a sensor, converts the captured biometric sound into electric signals, and outputs the electric signals to, for example, a speaker or an earpiece. To more securely capture biometric sound, an electronic stethoscope may remove noise such as ambient noise.

For example, Japanese Unexamined Patent Application Publication No. 2016-77623 describes an electronic stethoscope that includes a cover member having an opening that reduces noise introduced into a biometric sound sensor with physical stimulation.

### BRIEF SUMMARY OF THE DISCLOSURE

The electronic stethoscope described in Japanese Unexamined Patent Application Publication No. 2016-77623 still has room for improvement in noise reduction.

Accordingly, it is a possible benefit of the present disclosure to provide an electronic stethoscope capable of reducing noise.

An electronic stethoscope according to an aspect of the present disclosure is an electronic stethoscope that captures biometric sound, including: a sensor element that captures biometric sound; and a first housing that accommodates the sensor element, that has a pipe shape having a first end and a second end, and that receives a shielding member at the first end, wherein the first housing includes a first flow path that extends through the first housing and that connects a side of an inner wall and a side of an outer wall of the first housing to each other, wherein, around the sensor element, a NULL area in which sensitivity of the sensor element is reduced by greater than or equal to a predetermined value with respect to a maximum gain at which the sensitivity of the sensor element is maximum is defined, and wherein at least one of an opening of the first flow path formed in the inner wall of the first housing or an opening of the first flow path formed in the outer wall of the first housing is located in the NULL area of the sensor element.

The present disclosure can provide an electronic stethoscope capable of reducing noise.

### BRIEF DESCRIPTION OF THE SEVERAL VIEWS OF THE DRAWINGS

FIG. 1 is a schematic perspective view of an electronic stethoscope according to a first embodiment of the present disclosure;

FIG. 2 is a cross-sectional view taken along line A-A in FIG. 1;

FIG. 3 is a schematic diagram of directivity of a sensor element according to the first embodiment;

FIG. 4 is a diagram of an example of a measurement circuit for measuring directivity of the sensor element;

FIG. 5 is a diagram illustrating the position of a first flow path in the electronic stethoscope illustrated in FIG. 1;

FIG. 6 is a schematic cross-sectional view of an electronic stethoscope according to a first modification example of the first embodiment;

FIG. 7 is a schematic cross-sectional view of an electronic stethoscope according to a second modification example of the first embodiment;

FIG. 8 is a schematic cross-sectional view of an electronic stethoscope according to a second embodiment;

FIG. 9 is a schematic perspective view of an electronic stethoscope according to a third embodiment;

FIG. 10 is a cross-sectional view taken along line B-B in FIG. 9;

FIG. 11 is a schematic diagram of directivity of a sensor element according to the third embodiment;

FIG. 12 is a schematic cross-sectional view of an electronic stethoscope according to a modification example of the third embodiment;

FIG. 13 is a schematic cross-sectional view of an electronic stethoscope according to a fourth embodiment;

FIG. 14 is a schematic diagram of directivity of a sensor element according to the fourth embodiment;

FIG. 15 is a schematic cross-sectional view of an electronic stethoscope according to a fifth embodiment; and

FIG. 16 is a schematic cross-sectional view of an electronic stethoscope according to a modification example of the fifth embodiment.

### DETAILED DESCRIPTION OF THE DISCLOSURE

### Background Leading to Present Disclosure

A known electronic stethoscope converts biometric sound into electric signals, and outputs the signals to, for example, a speaker or an earpiece. The electronic stethoscope can amplify biometric sound with a small sound pressure level to improve auscultation accuracy.

During auscultation using an electronic stethoscope, various types of noise such as ambient noise, noise resulting from pressure fluctuation of the stethoscope, or noise resulting from contact pressure fluctuation caused by friction between the stethoscope and the patient's clothes may be introduced into a sensor of the electronic stethoscope as noise. To remove noise other than intended biometric sound, for example, Japanese Unexamined Patent Application Publication No. 2016-77623 describes an electronic stethoscope that includes a cover member having an opening that reduces noise introduced into a biometric sound sensor by physical stimulation.

However, the opening formed in the cover member is more likely to introduce, for example, ambient noise into the electronic stethoscope and to reduce auscultation accuracy.

Thus, the present inventor(s) studied an electronic stethoscope capable of reducing introduction of noise resulting from pressure fluctuation and ambient noise into the sensor element and improving auscultation accuracy, and achieved the disclosure described below.

Embodiments of the present disclosure are described below with reference to the attached drawings. Throughout the drawings, for ease of illustration, components are illustrated in an exaggerated manner.

Herein, the terms such as "first" or "second" are used solely for purposes of description and should not be construed as indicating or implying relative importance or an order of technical features. Features designated as "first" and "second" are not limited to a single instance of such features but may include one or more of them.

### First Embodiment

### Overall Structure

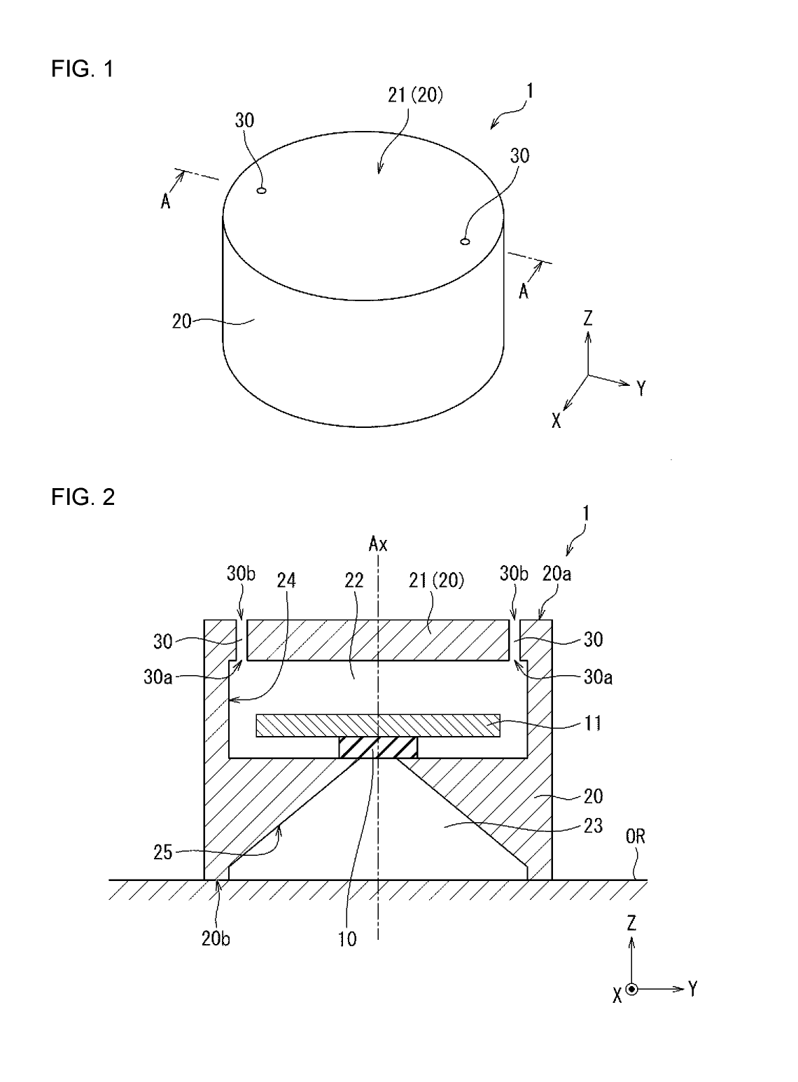

FIG. 1 is a schematic perspective view of an electronic stethoscope 1 according to a first embodiment of the present disclosure. FIG. 2 is a cross-sectional view taken along line A-A in FIG. 1. X-Y-Z orthogonal coordinate systems illustrated in the drawings are provided for ease of understanding the embodiments of the present disclosure, and not intended to limit the embodiments. The X-axis direction indicates the depth direction of an electronic stethoscope, the Y-axis direction indicates the width direction of the electronic stethoscope, and the Z-axis direction indicates the thickness direction of the electronic stethoscope. The Z-axis direction is a direction in which the electronic stethoscope comes into contact with a living body.

The electronic stethoscope 1 is an electronic device that picks up biometric sound produced by a living body such as a human body while being in contact with the living body. Examples of biometric sound include heartbeats, pulmonary sound, bloodstream sound, and bowel sound.

As illustrated in FIG. 1 and FIG. 2, the electronic stethoscope 1 includes a sensor element 10 and a first housing 20.

### Sensor Element

The sensor element 10 is a sensor that captures biometric sound and converts the biometric sound into electric signals. As illustrated in FIG. 2, the sensor element 10 is mounted on a substrate 11 and accommodated in the first housing 20 together with the substrate 11.

The sensor element 10 is formed from, for example, a microphone or a piezoelectric vibration plate. In the present embodiment described below, an electric condenser microphone is used as an example of the sensor element 10.

In some area around the sensor element 10 formed from, for example, a microphone or a piezoelectric vibration plate, the sensor element 10 has lower sensitivity (gain) than in other areas. Thus, a NULL area, in which the sensor element 10 has sensitivity further reduced by greater than or equal to a predetermined value than a maximum gain at which the sensor element 10 has maximum sensitivity, is defined around the sensor element 10.

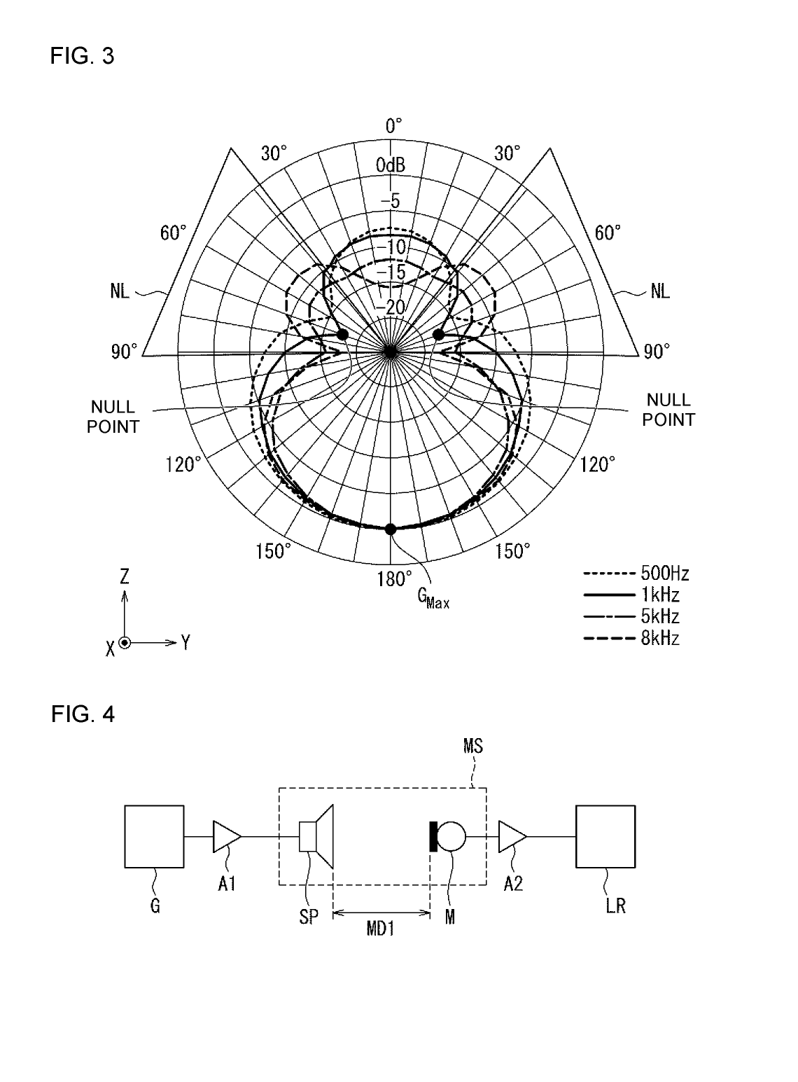

FIG. 3 is a schematic diagram of directivity of the sensor element 10 according to the first embodiment. The NULL area is described with reference to FIG. 3.

FIG. 3 illustrates distribution of sensitivity of the sensor element 10 disposed at the center of the graph and viewed in the X-axis direction. When the sensor element 10 is disposed at the center of the graph illustrated in FIG. 3, the position of the sensor element 10 in the -Z direction, that is, the position at 0 degrees in FIG. 3 corresponds to a maximum gain G_{Max}. FIG. 3 illustrates how much sensitivity of the sensor element 10 is reduced with respect to the maximum gain G_{Max} in various directions from the sensor element 10 at the center. For example, for the sensitivity to the sound of 1 kHz, the reduction with respect to the maximum gain G_{Max} is approximately -18 dB, which is the maximum, at the position ±110 degrees from the maximum gain G_{Max}. In the example of 1 kHz illustrated in FIG. 3, the positions ±110 degrees from the maximum gain G_{Max} are defined as NULL points. The NULL points are positions where the reduction of sensitivity with respect to the maximum gain G_{Max} is, for example, greater than or equal to 15 dB, and where the reduction of sensitivity with respect to the maximum gain G_{Max} is the maximum. Around the sensor element 10, areas around the NULL points, or areas defined by two triangles in FIG. 3 may be defined as NULL areas NL. The NULL areas NL are located around the sensor element 10, and are areas where the sensitivity of the sensor element 10 is reduced by greater than or equal to a predetermined value from the maximum gain G_{Max} where the sensitivity of the sensor element 10 is maximum. The predetermined value is, for example, 10 dB, and in the present embodiment, the area where the sensitivity of the sensor element 10 is reduced by, for example, greater than or equal to 10 dB from the maximum gain is defined as a NULL area NL.

In the present embodiment, the sensor element 10 is installed in the first housing 20 to direct the maximum gain G_{Max} of the sensor element 10 toward a second end 20b or a living body.

In the NULL areas NL, the sound introduced into the sensor element 10 can be reduced. The sensor element 10 installed in consideration of the NULL areas NL can reduce ambient noise or interference from unwanted sound sources.

In the present embodiment, as described later, the electronic stethoscope 1 with reduced noise can be provided by designing, for example, the shape or the structure of the first housing 20 in consideration of the NULL areas NL.

A method for measuring directivity of the sensor element 10 is described. FIG. 4 is a diagram of an example of a measurement circuit for measuring directivity of the sensor element 10. The measurement circuit in FIG. 4 includes a sinusoidal generator G, a driving amplifier A1, a microphone amplifier A2, a measurement sound source SP, a measurement target microphone M, and a level recorder LR. The measurement target microphone M is positioned at a distance MD1 of 50 cm from the measurement sound source SP, and a reference axis of the measurement sound source and a reference axis of the measurement target microphone M are aligned. The reference axis corresponds to a measurement sound incidence direction. Using the measurement circuit illustrated in FIG. 4, the reference axis is rotated and the sensitivity level of the measurement target microphone M is measured at various angles. The sensitivity level measurement includes measurement of the sound pressure level captured by the measurement target microphone M with the level recorder LR. By comparing the sound pressure levels at various angles, the positions corresponding to the maximum gain G_{Max} and the positions corresponding to the NULL points can be specified.

Directivity of the sensor element 10 is measured in accordance with JIS C5502 standards.

Measurement is performed at a normal temperature (higher than or equal to 5°C and lower than or equal to 35°C), a normal humidity (relative humidity higher than or equal to 45% and lower than or equal to 85%), and an atmospheric pressure higher than or equal to 86 kPa and lower than or equal to 106 kPa (temperature grade 15 and humidity grade 20 in JIS-Z8703).

Generally, a space regarded as a free sound field is used for measurement, and the sound field characteristics of the frequency range from higher than or equal to 100 Hz to lower than or equal to 10 kHz have deviation from the free sound field characteristics at the measurement point not exceeding ±1 dB. However, unless no doubt arises regarding the determination, the space with equivalent characteristics may be used. An anechoic room satisfying these conditions for the measurement space is used for measurement.

The reference point of a microphone under test (measurement target microphone M) is defined 50 cm apart from the reference point of a measurement sound source, the reference axis of the microphone and the reference axis of the measurement sound source are aligned, and the microphone is positioned to face the sound source. This state is defined as a reference measurement state. As the reference measurement state during measurement while the sound pressure is adjusted to be uniform by the measurement microphone or in comparison with the measurement microphone, the reference points of the measurement microphone and the microphone under test are defined on both sides of the reference axis of the measurement sound source at a position 50 cm apart from the measurement sound source where the microphone under test is to be positioned, and the measurement microphone and the microphone under test are positioned to face the measurement sound source to arrange their reference axes in parallel to the reference axis of the measurement sound source. In addition, when the microphone is a microphone for use in vicinity of a sound source, the directivity is measured in the measurement states in accordance with the maintained usage conditions.

The measurement space is to have no background noise that causes errors exceeding ±1 dB in the measurement results. For measurement of intrinsic noise, a sound insulation box satisfying the conditions regarding disorders caused by background noise is used.

Unless otherwise specified, the measurement frequency range is from higher than or equal to 20 Hz and lower than or equal to 20 kHz.

The measurement microphone is generally a standard condenser microphone defined in JIS C5515. However, unless no doubt arises regarding the measurement, a pressure microphone formed from a standard condenser microphone in JIS C5515 may be used.

The measurement sound source satisfies the following conditions. (1) The sound pressure level at the measurement position in the reference measurement state is substantially uniform within the measurement frequency range, and is axially symmetric within ±1 dB. (2) The sound pressure frequency has flat characteristic in accordance with the control performance of a voltage controller. (3) A distortion factor is less than or equal to 1% throughout the full measurement frequency range.

The frequency change speed of an automatic frequency characteristic recording device used for measurement does not fluctuate a measurement value over ±1 dB as compared to a measurement value in a stationary state.

A directivity pattern is measured by installing the measurement circuit in FIG. 4, and rotating the reference axis about the reference point of the microphone under test (measurement target microphone M) installed in the reference measurement state in a horizontal direction or a vertical direction while changing the rotation angle point by point or continuously by 0 degrees to 360 degrees. At this time, at least three designated frequencies including 1 kHz are selected from among 125 Hz, 250 Hz, 500 Hz, 1 kHz, 2 kHz, 4 kHz, 8 kHz, and 16 kHz. The difference between the sensitivity level corresponding to the rotation angle and the front sensitivity level is obtained, and recorded. The recording may be performed manually or using an automatic recording device. The measurement frequency is specified by a manufacturer.

As illustrated in FIG. 3, the NULL points and NULL areas may vary depending on the sound frequency. Thus, the NULL areas NL may be defined as appropriate by, for example, the frequency of intended biometric sound.

### First Housing

The first housing 20 is a so-called chestpiece of the electronic stethoscope 1, and is a portion held by a user such as a doctor during use of the electronic stethoscope 1.

The first housing 20 has a pipe shape having a first end 20a and a second end 20b. In the present embodiment, as illustrated in FIG. 2, the first housing 20 has a cylinder shape having an axis AX parallel to a Z axis. A shielding member 21 is disposed at the first end 20a of the first housing 20. In the present embodiment, the shielding member 21 is integrated with the first housing 20. More specifically, the shielding member 21 is a part of the first housing 20. The shielding member 21 may be separate from the first housing 20.

In the present embodiment, the first housing 20 has a first space 22 located closer to the first end 20a than the sensor element 10, and a second space 23 located closer to the second end 20b than the sensor element 10. As illustrated in FIG. 2, the first space 22 is a space defined by the shielding member 21 and a first wall portion 24. The second space 23 is a space defined by a second wall portion 25. In other words, the first housing 20 includes the first wall portion 24 that defines the first space 22 together with the shielding member 21, and the second wall portion 25 that defines the second space. The first housing 20 may have a structure including only one space continuous from the first end 20a to the second end 20b.

The first space 22 can accommodate, for example, the sensor element 10 and the substrate 11 on which the sensor element 10 is mounted. The first space 22 is, for example, a cylindrical space. The second space 23 is a space for amplifying sound from a living body. The second space 23 has, for example, a truncated cone shape. As illustrated in FIG. 2, the second space 23 has a truncated cone shape with the second wall portion 25 gradually expanding from the sensor element 10 toward the second end 20b of the first housing 20.

As illustrated in FIG. 2, the first housing 20 includes first flow paths 30. The first flow paths 30 are flow paths that connect the inner wall side and the outer wall side of the first housing 20. In other words, the first flow paths 30 are flow paths that extend through the first housing 20 to connect the inner wall side of the first housing 20 and the outer wall side of the first housing 20. In the present embodiment, the first flow paths 30 connect the first space 22 of the first housing 20 and the outside of the first housing 20. In the present embodiment, the first flow paths 30 are located at positions not to overlap the sensor element 10 and the substrate 11 when the sensor element 10 is viewed in the vertical direction (Z-axis direction) and the horizontal direction (X-axis direction or Y-axis direction).

As illustrated in FIG. 1 and FIG. 2, in the present embodiment, the first housing 20 includes two first flow paths 30. The first flow paths 30 extend in a direction (Z direction) from the first end 20a toward the second end 20b. Each first flow path 30 includes a first opening 30a located inside the first housing 20, and a second opening 30b located outside the first housing 20.

As illustrated in FIG. 2, when the electronic stethoscope 1 is in use, the second end 20b of the first housing 20 is pressed against a living body OR. At this time, the internal space including the first space 22 and the second space 23 of the first housing 20 is hermetically sealed by the first wall portion 24, the second wall portion 25, and the shielding member 21 of the first housing 20, and the living body OR. When the internal space of the first housing 20 is hermetically sealed, the sensor element 10 may detect noise due to, for example, a change of pressure on the electronic stethoscope 1. In the present embodiment, the first housing 20 has the first flow paths 30, and thus can release the pressure in the internal space of the hermetically sealed first housing 20 to the outside of the first housing 20. Releasing the pressure in the internal space of the first housing 20 to the outside can reduce noise caused by, for example, a change of pressure in the internal space of the first housing 20.

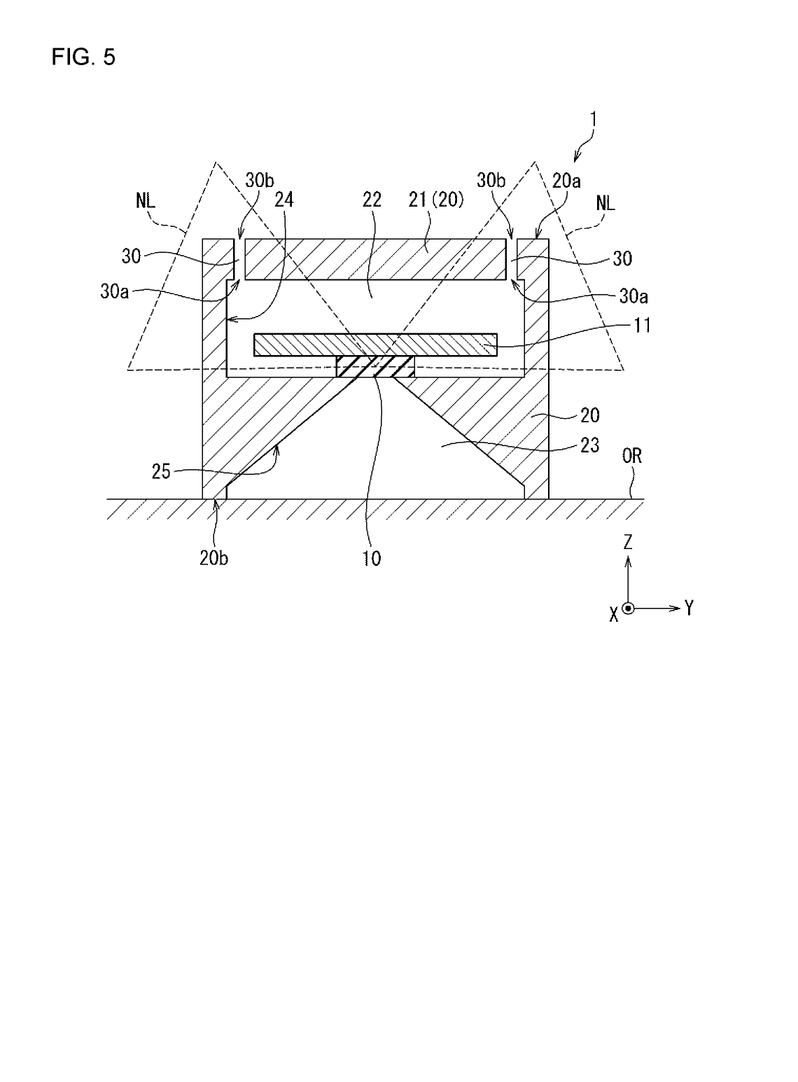

FIG. 5 is a diagram illustrating the positions of the first flow paths 30 in the electronic stethoscope 1 in FIG. 1. FIG. 5 illustrates the NULL areas NL of the sensor element 10 in FIG. 3 and the cross-sectional view of the electronic stethoscope 1 in FIG. 2 in an overlapping manner.

As illustrated in FIG. 5, the first flow paths 30 are located to be included in the NULL areas NL of the sensor element 10. When the first flow paths 30 are included in the NULL areas NL, noise such as noise from the outside introduced from the first flow paths 30 into the sensor element 10 can be reduced. When at least one of the first opening 30a and the second opening 30b of each first flow path 30 is located in the NULL areas NL of the sensor element 10, noise from the outside introduced into the sensor element 10 can be reduced.

### Effects

The above embodiments can exert effects described below.

The electronic stethoscope 1 is an electronic stethoscope that captures biometric sound, and includes the sensor element 10 and the first housing 20. The sensor element 10 is a sensor that captures biometric sound. The first housing 20 is a housing for accommodating the sensor element 10. The first housing 20 has a pipe shape having the first end 20a and the second end 20b. The first housing 20 receives the shielding member 21 at the first end 20a. The first housing 20 includes the first flow paths 30 that connect an inside and an outside of the first housing 20. Around the sensor element 10, NULL areas NL in which the sensitivity of the sensor element 10 is reduced by greater than or equal to a predetermined value with respect to a maximum gain G_{Max} at which the sensitivity of the sensor element 10 is maximum are defined. Of each first flow path 30, at least one of the opening 30a located inside the first housing 20 or the opening 30b located outside the first housing 20 is located in the NULL areas NL of the sensor element 10.

The electronic stethoscope 1 with this structure can thus reduce noise. The electronic stethoscope 1 including the first flow paths 30 can reduce noise produced by pressure fluctuation caused when the internal space of the first housing 20 is hermetically sealed while the electronic stethoscope 1 is brought into contact with a living body OR for auscultation. When the first flow paths 30 are disposed in the NULL areas NL, the sensitivity of the sensor element 10 to noise introduced into the inside of the first housing 20 through the first flow paths 30 can be reduced. Thus, the electronic stethoscope 1 that reduces noise can be provided.

The first housing 20 includes the first wall portion 24 that defines the first space 22, located closer to the first end 20a than the sensor element 10, together with the shielding member 21, and the second wall portion 25 that defines the second space 23, located closer to the second end 20b than the sensor element 10.

The electronic stethoscope 1 with this structure can amplify the biometric sound in the second space 23, and thus enables highly accurate auscultation.

The first flow paths 30 connect the first space 22 and the outside of the first housing 20.

The electronic stethoscope 1 with this structure can further efficiently reduce noise from the outside.

The NULL areas NL are areas where the sensitivity of the sensor element 10 is reduced by greater than or equal to 10 dB than the maximum gain G_{Max}.

The electronic stethoscope 1 with this structure can appropriately set the NULL areas NL.

In the embodiment described above as an example, the first housing 20 includes two first flow paths 30, but the present disclosure is not limited to this example. The first housing 20 may include one first flow path 30 or three or more first flow paths 30.

### Modification Example

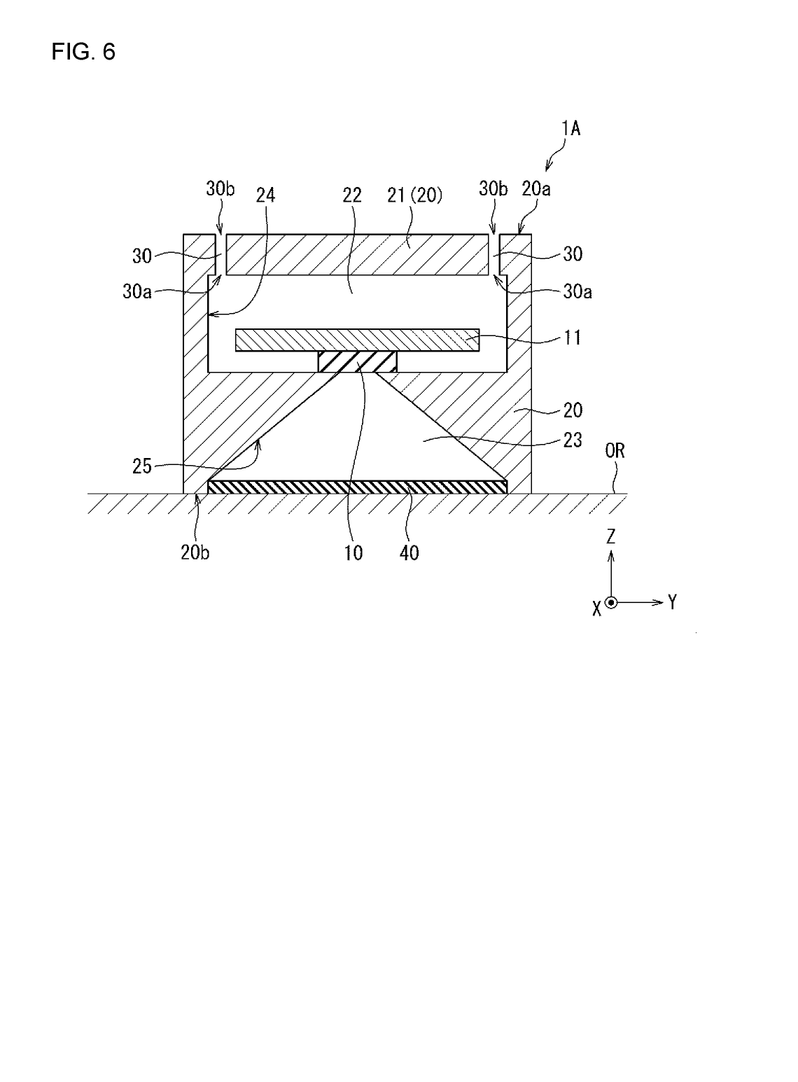

FIG. 6 is a schematic cross-sectional view of an electronic stethoscope 1A according to a first modification example of the first embodiment. As illustrated in FIG. 6, a diaphragm 40 may be disposed at the second end 20b of the first housing 20. When the electronic stethoscope 1A is used for auscultation, the diaphragm 40 is brought into contact with the living body OR, and vibrates at the frequency and the number of vibrations corresponding to the biometric sound. The diaphragm 40 is a flexible sheet member, and formed from a material such as glass epoxy.

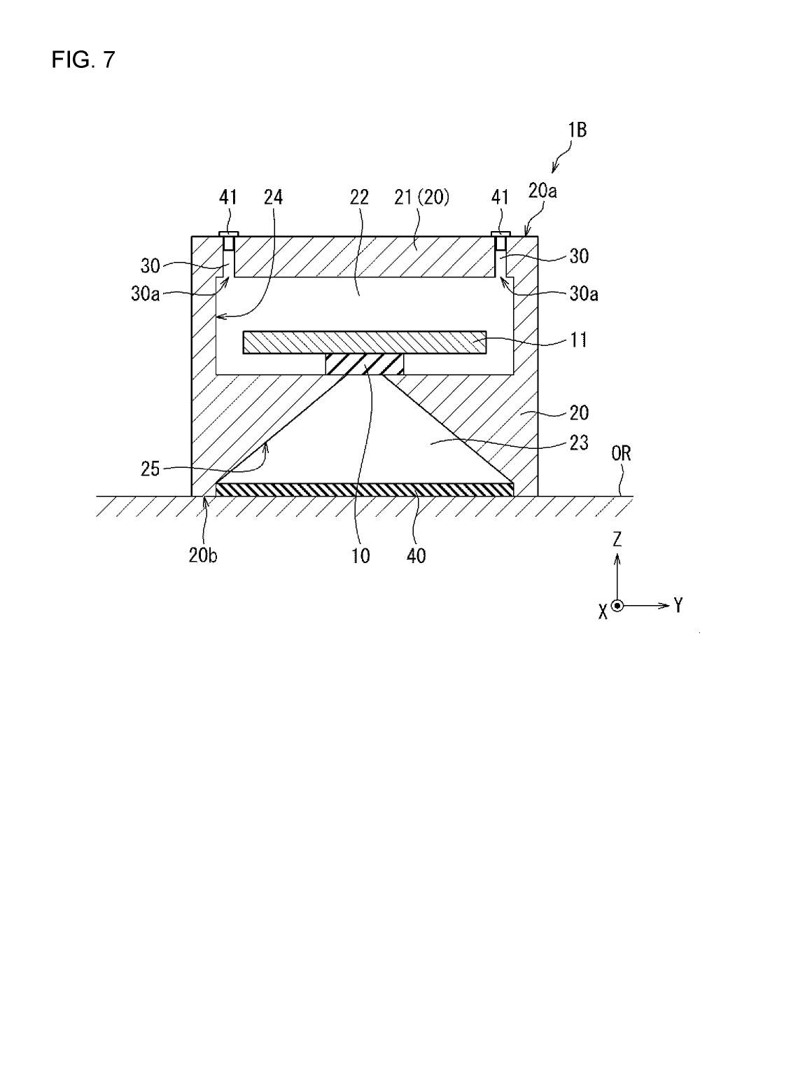

FIG. 7 is a schematic cross-sectional view of an electronic stethoscope 1B according to a second modification example of the first embodiment. As illustrated in FIG. 7, screws 41 may be disposed at the first flow paths 30. For example, when the first housing 20 and the shielding member 21 are formed from separate components, the first flow paths 30 can be used as threaded holes, and the first housing 20 and the shielding member 21 can be fixed with the screws 41. Alternatively, the first flow paths 30 can be used as holes for threading the first housing 20 with cables connected to, for example, the substrate 11. When, for example, the screws 41 or the cables are to be disposed in the first flow paths 30, to secure breathability from the inside to the outside of the first housing 20, the screws 41 or the cables may have a diameter smaller than the inner diameter of the first flow paths 30. When the first flow paths 30 are used as holes to receive, for example, the screws 41 or the cables, the electronic stethoscope 1A can reduce noise while reducing the manufacturing costs. Appropriately selecting the material of, for example, the screws 41 or the cables can improve the reduction effects of noise introduced from the outside as compared to the structure simply including the first flow paths 30.

### Second Embodiment

An electronic stethoscope according to a second embodiment of the present disclosure is described. The electronic stethoscope according to the second embodiment is mainly described in relation to the points different from the electronic stethoscope according to the first embodiment. In the second embodiment, the same components or equivalent components as those in the first embodiment are denoted with the same reference signs. In the second embodiment, the same description as that in the first embodiment is omitted.

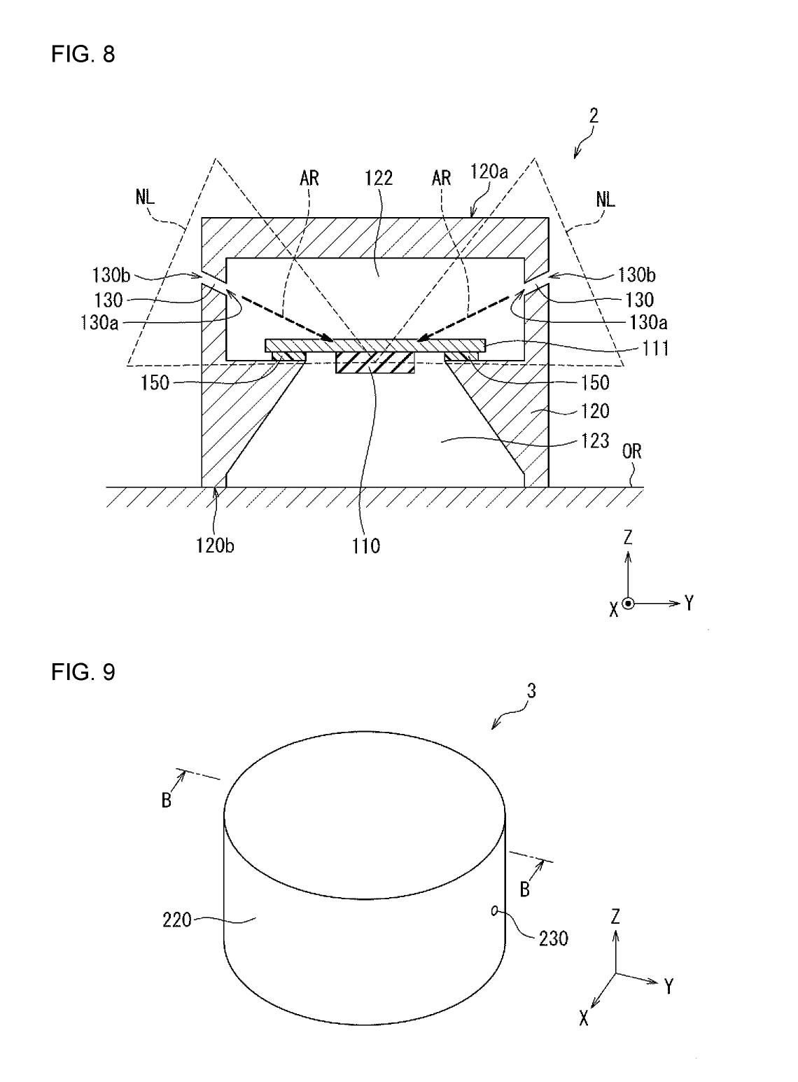

FIG. 8 is a schematic cross-sectional view of an electronic stethoscope 2 according to the second embodiment. As illustrated in FIG. 8, the second embodiment differs from the first embodiment in that an electronic stethoscope 2 includes a venting member 150 that separates a first space 122 and a second space 123 from each other with a substrate 111, and that allows air to flow therethrough from the second space 123 to the first space 122. The second embodiment differs from the first embodiment in that first flow paths 130 extend rectilinearly in a direction toward the sensor element 110 in the NULL areas NL.

In the present embodiment, the substrate 111 connected to a sensor element 110 is disposed to separate the first space 122 and the second space 123 from each other. Inside a first housing 120, the venting member 150 is disposed to be held between the substrate 111 and the first housing 120. More specifically, the venting member 150 separates the first space 122 and the second space 123 from each other together with the substrate 111.

The venting member 150 is formed from a component such as a sponge or a washer. The venting member 150 allows air to flow from the second space 123 to the first space 122. Thus, providing the venting member 150 can reduce noise caused by pressure fluctuation while further improving airtightness in the second space 123. The electronic stethoscope 2 thus enables auscultation with higher accuracy.

As illustrated in FIG. 8, the venting member 150 is disposed in the NULL areas NL of the sensor element 110. As described above, the venting member 150 allows air to pass therethrough. Installing the venting member 150 in the NULL areas NL can reduce outside noise introduced from the venting member 150 into the sensor element 110.

In the present embodiment, the first flow paths 130 extend rectilinearly in a direction toward the sensor element 110 in the NULL areas NL. As illustrated in FIG. 8, the first flow paths 130 are disposed in the NULL areas NL along arrows AR indicating the directions toward the sensor element 110.

### Third Embodiment

An electronic stethoscope according to a third embodiment of the present disclosure is described. The electronic stethoscope according to the third embodiment is described mainly in relation to the points different from the electronic stethoscope according to the second embodiment. In the third embodiment, the same components or equivalent components as those in the second embodiment are denoted with the same reference signs. In the third embodiment, the same description as that in the second embodiment is omitted.

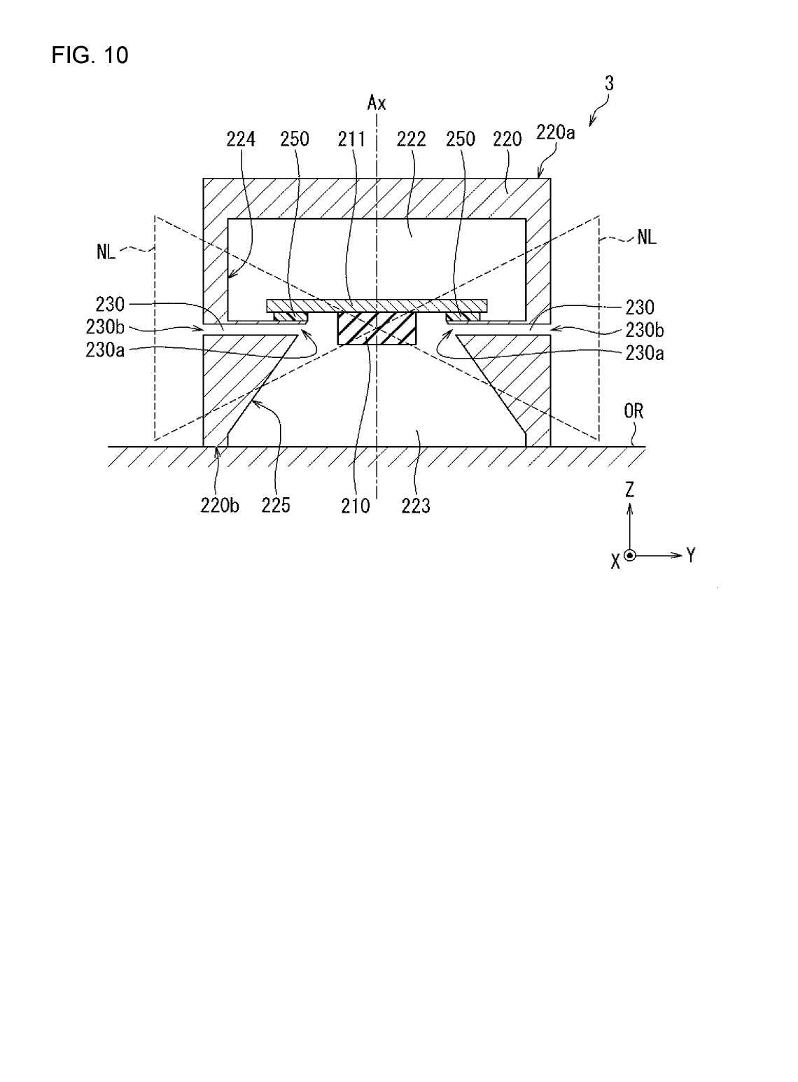

FIG. 9 is a schematic perspective view of an electronic stethoscope 3 according to a third embodiment. FIG. 10 is a cross-sectional view taken along line B-B in FIG. 9. As illustrated in FIG. 9 and FIG. 10, the third embodiment differs from the second embodiment in that first flow paths 230 extend rectilinearly in the vertical direction with respect to a center axis direction AX of a first housing 220.

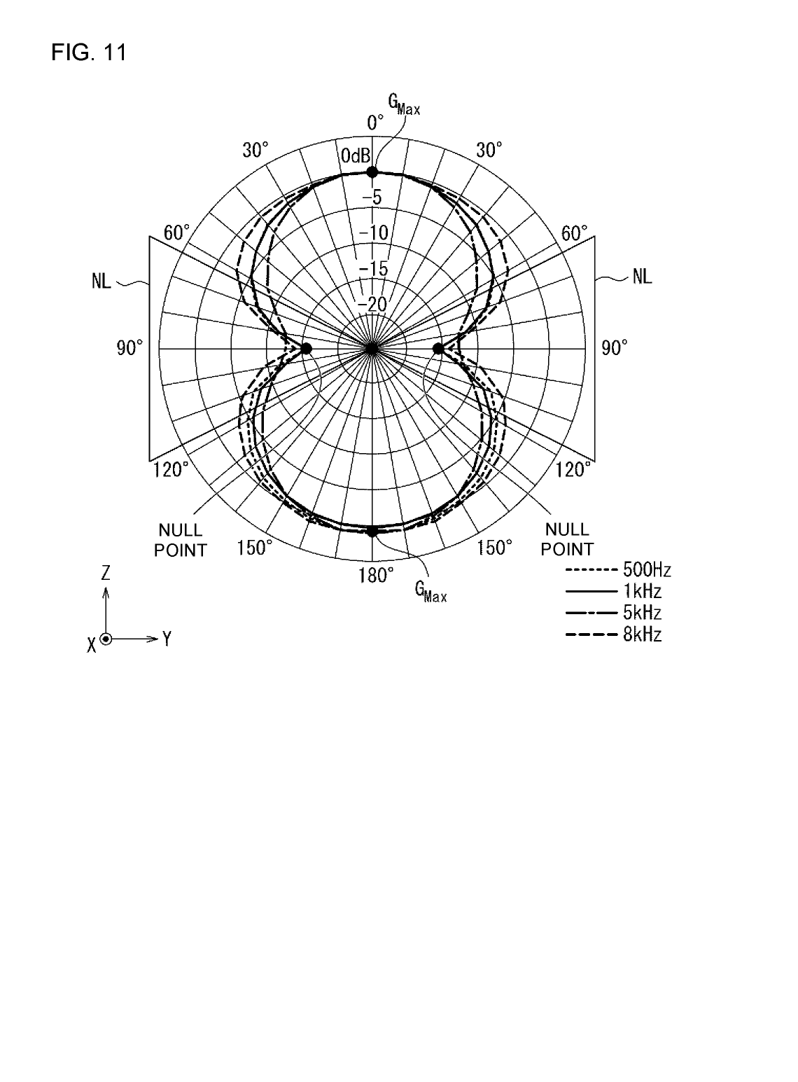

FIG. 11 is a schematic diagram of directivity of a sensor element 210 according to a third embodiment. FIG. 11 illustrates distribution of sensitivity of the sensor element 210 disposed at the center of the graph, viewed in the X-axis direction. In the present embodiment, the sensor element 210 has maximum sensitivity at two positions in the Z-axis direction from the sensor element 210. More specifically, the maximum gain G_{Max} of the sensor element 210 is located at a 0-degree position and a 180-degree position illustrated in FIG. 11. As illustrated in FIG. 11, positions ±90 degrees around the sensor element 210 are defined as NULL points. As illustrated in FIG. 11, in the sensor element 210 according to the present embodiment, the NULL points are located at positions ±90 degrees in each frequency. Thus, the electronic stethoscope 3 can be designed independently from the frequency. The NULL areas NL of the sensor element 210 extend in a direction orthogonal to an axis direction AX of the first housing 20 when viewed from the sensor element 210.

In the present embodiment, as illustrated in FIG. 10, the first flow paths 230 extend rectilinearly in a direction orthogonal to the center axis direction AX of the first housing 220. In other words, in each first flow path 230, the direction from a first opening 230a located inside the first housing 220 toward a second opening 230b located outside the first housing 220 is along the substrate surface of a substrate 211 to which the sensor element 210 is connected. The first flow paths 230 connect a second space 223 and the outside of the first housing 220.

As in the case of the second embodiment, the substrate 211 separates a first space 222 and the second space 223 from each other with a venting member 250. The electronic stethoscope 3 according to the third embodiment can thus release the pressure in the hermetically sealed second space 223 to the outside of the first housing 220 through the first flow paths 230 by bringing a second end 220b of the first housing 220 into contact with the living body OR during auscultation. The electronic stethoscope 3 can also release the pressure in the second space 223 to the first space 222 through the venting member 250.

The electronic stethoscope 3 with this structure can directly release the pressure to the outside of the first housing 220 from the hermetically sealed second space 223 during use of the electronic stethoscope 3. When the first flow paths 230 are formed to extend in the direction orthogonal to the center axis direction AX of the first housing 220, the electronic stethoscope 3 has a simple structure. Thus, components included in the electronic stethoscope 3 can be designed, manufactured, and assembled at lower costs.

### Modification Example

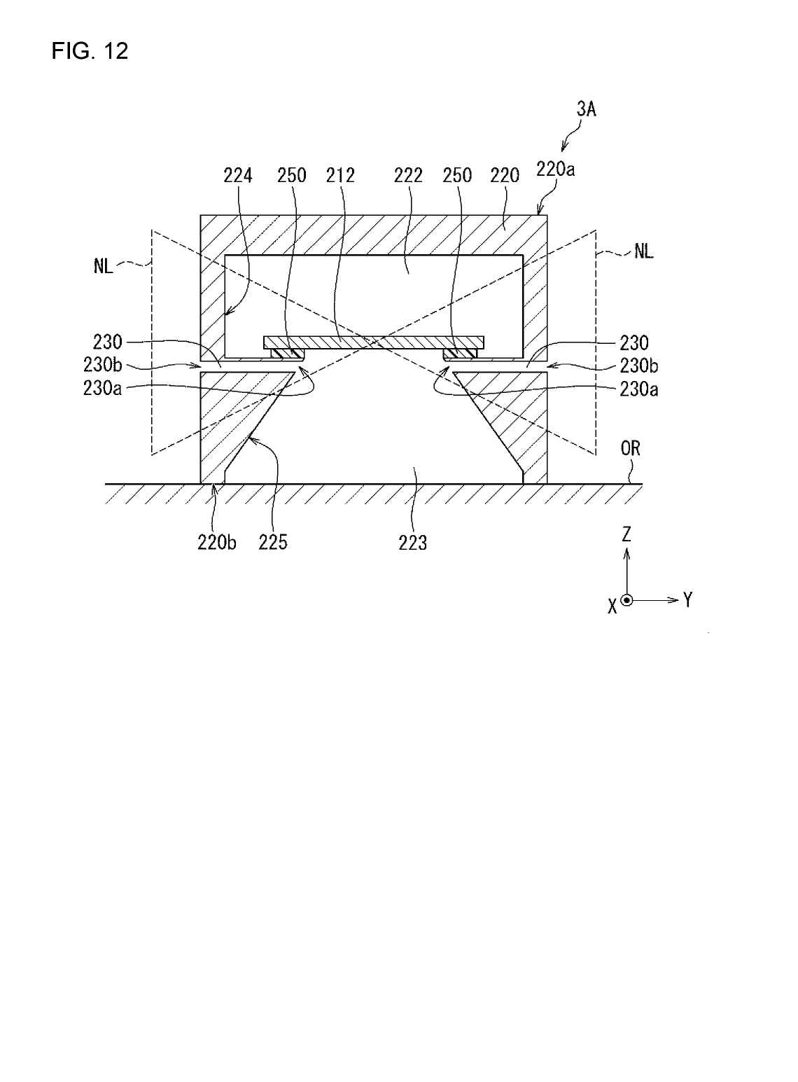

FIG. 12 is a schematic cross-sectional view of an electronic stethoscope 3A according to a modification example of the third embodiment. In the example illustrated in FIG. 12, a sensor element 212 is formed from a piezoelectric vibration plate. The electronic stethoscope 3A including the sensor element 212 formed from a piezoelectric vibration plate has a simple structure, and thus can be manufactured at lower costs.

### Fourth Embodiment

An electronic stethoscope according to a fourth embodiment of the present disclosure is described. The electronic stethoscope according to the fourth embodiment is described mainly in relation to the points different from the electronic stethoscope according to the second embodiment. In the fourth embodiment, the same components or equivalent components as those in the second embodiment are denoted with the same reference signs. In the fourth embodiment, the same description as that in the second embodiment is omitted.

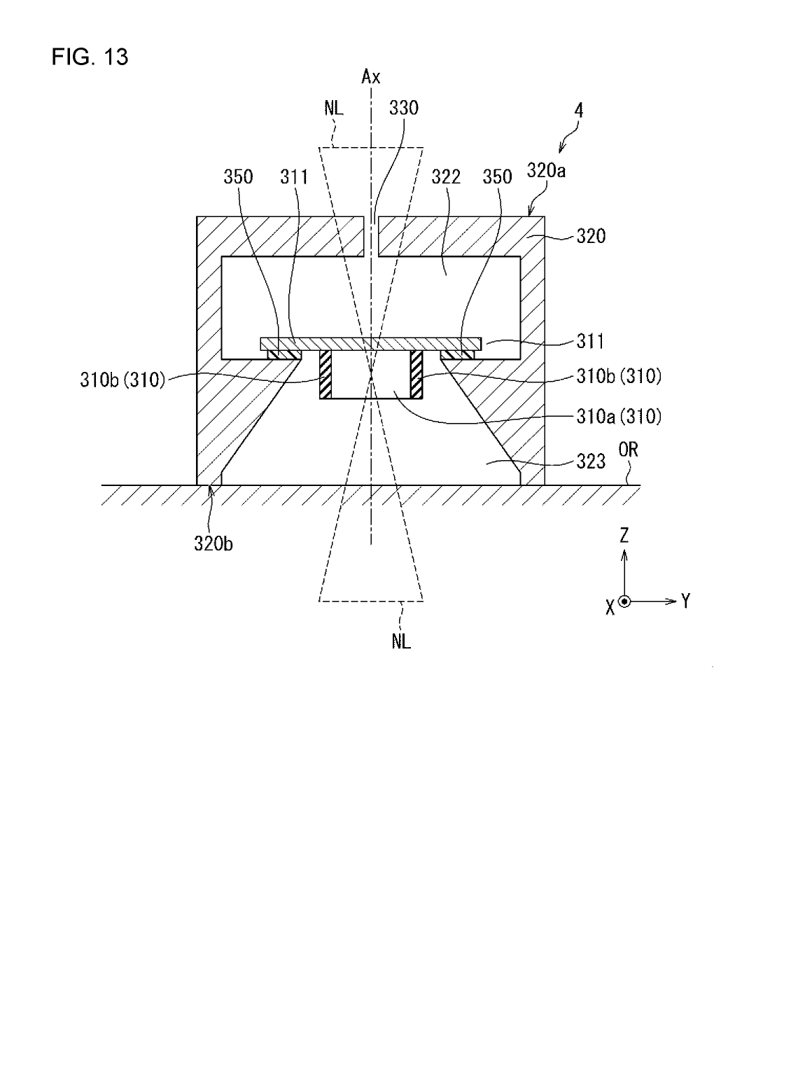

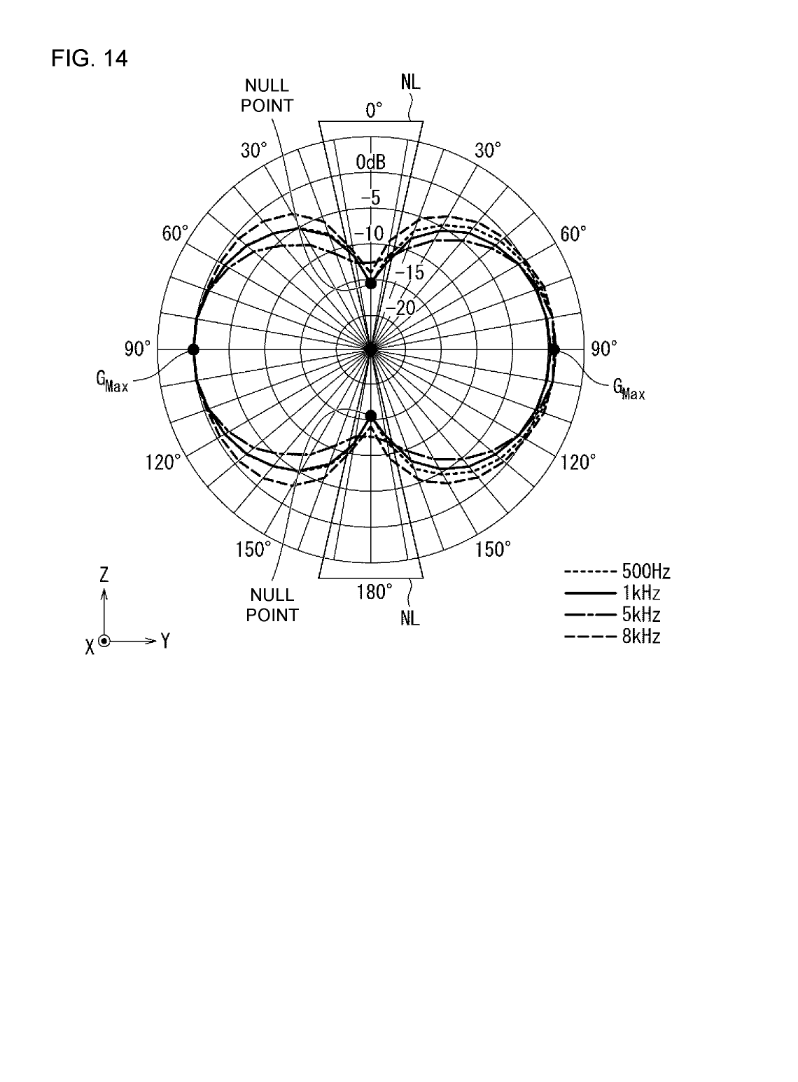

FIG. 13 is a schematic cross-sectional view of an electronic stethoscope 4 according to a fourth embodiment. FIG. 14 is a schematic diagram of directivity of a sensor element 310 according to the fourth embodiment. As illustrated in FIG. 13, the fourth embodiment differs from the second embodiment in that the sensor element 310 is formed from a cylindrical piezoelectric microphone. In addition, as illustrated in FIG. 14, the fourth embodiment differs from the second embodiment in the positions of NULL areas NL of the sensor element 310. In addition, the fourth embodiment differs from the second embodiment in the position of a first flow path 330.

As illustrated in FIG. 13, in the present embodiment, the sensor element 310 is formed from a cylindrical piezoelectric microphone. The cylindrical piezoelectric microphone includes a hollow cylinder 310a and a piezoelectric device 310b wound around a side wall of the hollow cylinder 310a. The sensor element 310 is mounted on a substrate 311 that holds the sensor element 310. As in the case of the second embodiment, the substrate 311 separates a first space 322 and a second space 323 of a first housing 320 from each other together with a venting member 350.

When the sensor element 310 is formed from a cylindrical piezoelectric microphone, as illustrated in FIG. 14, the maximum gain G_{Max} is located at two positions (at The positions at plus or minus 90 degrees position in FIG. 14) in the Y-axis direction from the sensor element 310. The maximum gain G_{Max} is located ±90 degrees position. As illustrated in FIG. 14, at positions ±60 degrees and ±120 degrees from the maximum gain G_{Max} at 0 degrees, the sensor element 310 has higher sensitivity as compared to when being at the NULL points. In the present embodiment, the positions where the sensor element 310 has relatively higher sensitivity but not corresponding to the maximum gain G_{Max} are located closer to a second end 320b of the first housing 320. In the present embodiment, the NULL areas NL extend in the direction parallel to the Z-axis direction. In other words, as illustrated in FIG. 13, the NULL areas NL extend perpendicularly along the central axis direction AX of the first housing 320 from the sensor element 310.

As illustrated in FIG. 13, in the present embodiment, the first flow path 330 extends rectilinearly in the center axis direction AX of the first housing 320. In other words, the first flow path 330 extends from the sensor element 310 toward a first end 320a of the first housing 320. In the present embodiment, the first housing 320 includes one first flow path 330. The electronic stethoscope 4 can thus be manufactured at lower costs than a structure having multiple first flow paths.

The electronic stethoscope 4 with this structure can reduce noise from a portion other than a portion that comes into contact with a living body. In addition, the electronic stethoscope 4 including one first flow path 330 can be manufactured at lower costs.

### Fifth Embodiment

An electronic stethoscope according to a fifth embodiment of the present disclosure is described. The electronic stethoscope according to the fifth embodiment is described mainly in relation to the points different from the electronic stethoscope according to the second embodiment. In the fifth embodiment, the same components or equivalent components as those in the second embodiment are denoted with the same reference signs. In the fifth embodiment, the same description as that in the second embodiment is omitted.

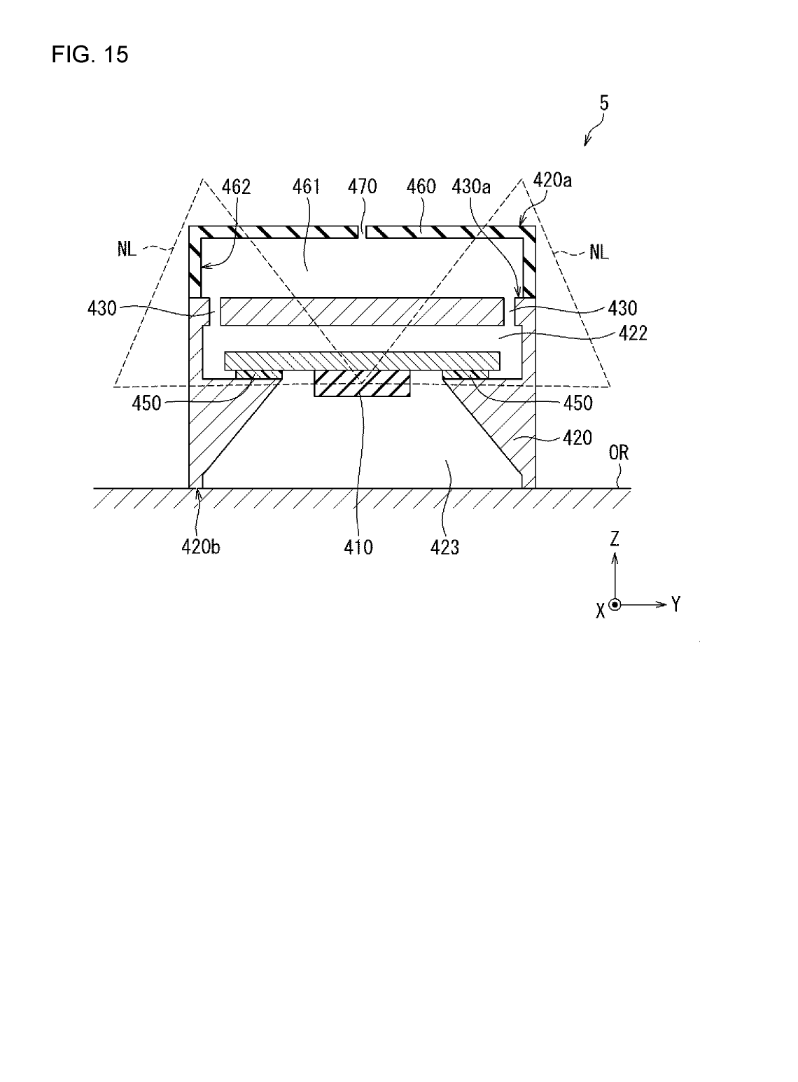

FIG. 15 is a schematic cross-sectional view of an electronic stethoscope 5 according to a fifth embodiment. As illustrated in FIG. 15, the fifth embodiment differs from the second embodiment in that the electronic stethoscope 5 further includes a second housing 460.

The second housing 460 is disposed closer to a first end 420a of a first housing 420. The second housing 460 includes a third wall portion 462 that defines a third space 461 adjacent to a first space 422 of the first housing 420. A display device (not illustrated) such as a display may be disposed in the second housing 460. For example, a substrate (not illustrated) connected to a display device such as a display may be accommodated in the third space 461. The second housing 460 is formed from a material different from the material of the first housing 420.

In the present embodiment, in addition to first flow paths 430 formed in the first housing 420, a second flow path 470 is formed in the second housing 460. The first flow paths 430 connect the first space 422 of the first housing 420 and the third space 461 of the second housing 460. The second flow path 470 connects the third space 461 and the outside of the electronic stethoscope 5 to each other.

In the present embodiment, a second space 423 is hermetically sealed when a second end 420b of the first housing 420 is brought into contact with the living body OR during auscultation. The pressure on the second space 423 can be released to the first space 222 through a venting member 450. In addition, the pressure can be released from the first space 422 to the third space 461 through the first flow paths 430. In addition, the pressure can be released from the third space 461 to the outside of the electronic stethoscope 5 through the second flow path 470.

The electronic stethoscope 5 according to the present embodiment includes two first flow paths 430 and one second flow path 470. The two first flow paths 430 are located in the NULL areas NL of a sensor element 410. The second flow path 470 is located outside the NULL areas NL.

In the present embodiment, noise introduced through the second flow path 470 connected with the outside of the electronic stethoscope 5 is introduced into the third space 461, passes through the first flow paths 430 from the third space, and arrives at the first space 422 and the sensor element 410. The first flow paths 430 are located in the NULL areas NL, and can thus reduce noise introduced into the sensor element 410.

The electronic stethoscope 5 with this structure can more efficiently reduce noise introduced from the NULL areas NL. The internal space of the first housing 420 and the outside of the electronic stethoscope 5 are connected via two stages of flow paths, including the first flow paths 430 and the second flow path 470. This structure can reduce noise introduced into the internal space of the first housing 420.

The electronic stethoscope 5 in which the second flow path 470 is disposed outside the NULL areas NL has improved design freedom. The electronic stethoscope 5 in which the second flow path 470 is disposed outside the NULL areas NL can retain the space in the second housing 460 for receiving, for example, buttons or a display.

### Modification Example

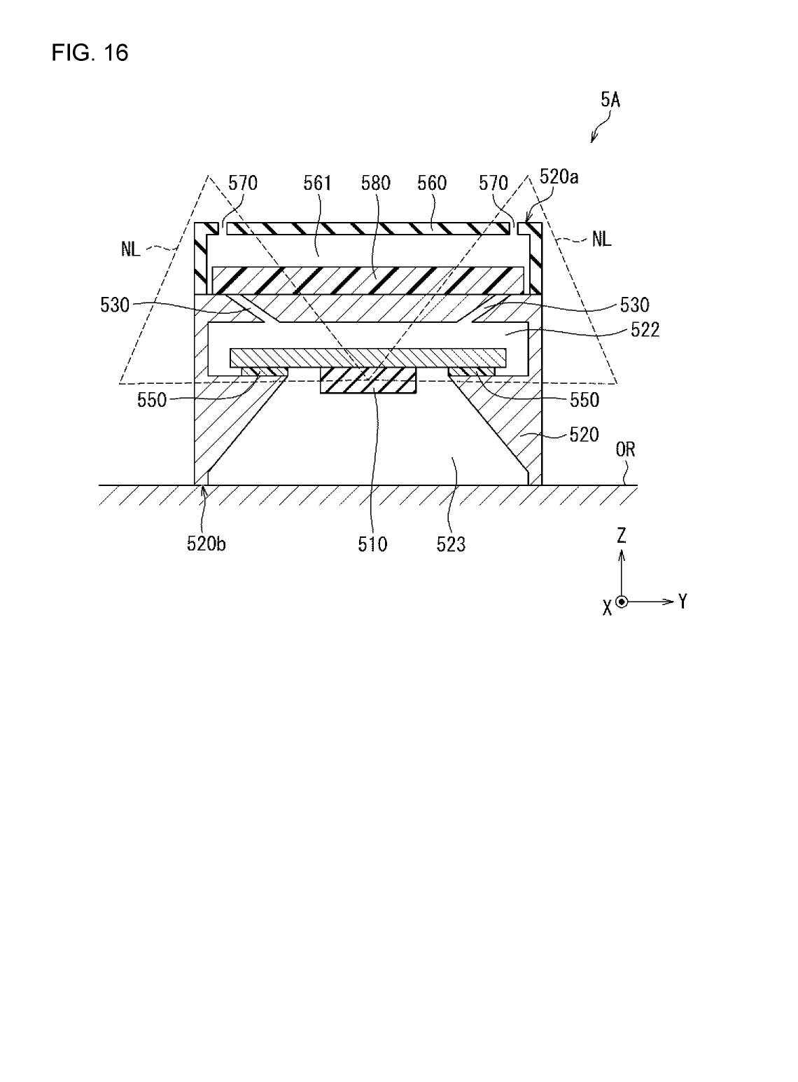

FIG. 16 is a schematic cross-sectional view of an electronic stethoscope 5A according to a modification example of the fifth embodiment. As illustrated in FIG. 16, a sound absorber 580 may be disposed in a third space 561 of a second housing 560. When disposed to block openings located outside a first housing 520 of a first flow path 530, the sound absorber 580 can reduce noise introduced from the third space 561 into a first space 522. A second flow path 570 formed in the second housing 560 may be disposed within the NULL areas NL of a sensor element 510, and extend in a direction crossing the first flow path 530. In the electronic stethoscope 5A with this structure, the second housing 560 can function as an acoustic buffer space, and the sound absorber can further reduce noise introduced into the first housing 520.

### Summarization of Embodiments

(1) An electronic stethoscope according to an aspect of the present disclosure is an electronic stethoscope that captures biometric sound, including: a sensor element that captures biometric sound; and a first housing that accommodates the sensor element, that has a pipe shape having a first end and a second end, and that receives a shielding member at the first end, wherein the first housing includes a first flow path that extends through the first housing and that connects a side of an inner wall and a side of an outer wall of the first housing to each other, wherein, around the sensor element, a NULL area in which sensitivity of the sensor element is reduced by greater than or equal to a predetermined value with respect to a maximum gain at which the sensitivity of the sensor element is maximum is defined, and wherein at least one of an opening of the first flow path formed in the inner wall of the first housing or an opening of the first flow path formed in the outer wall of the first housing is located in the NULL area of the sensor element.

(2) The electronic stethoscope according to (1), wherein the first flow path rectilinearly connects the side of the inner wall and the side of the outer wall to each other in the NULL area in a direction toward the sensor element.

(3) An electronic stethoscope according to an aspect of the present disclosure is an electronic stethoscope that captures biometric sound, including: a sensor element that captures biometric sound; and a first housing that accommodates the sensor element, that has a pipe shape having a first end and a second end, and that receives a shielding member at the first end, wherein the first housing includes a first flow path that extends through the first housing and that connects a side of an inner wall and a side of an outer wall of the first housing to each other, wherein, around the sensor element, a NULL area is defined to extend from the sensor element toward the first end, and wherein the first flow path rectilinearly connects the side of the inner wall and the side of the outer wall to each other in the NULL area in a center axis direction of the first housing.

(4) An electronic stethoscope according to an aspect of the present disclosure is an electronic stethoscope that captures biometric sound, including: a sensor element that captures biometric sound; and a first housing that accommodates the sensor element, that has a pipe shape having a first end and a second end, and that receives a shielding member at the first end, wherein the first housing includes a first flow path that extends through the first housing and that connects a side of an inner wall and a side of an outer wall of the first housing to each other, wherein, around the sensor element, a NULL area is defined in a direction orthogonal to a center axis direction of the first housing from the sensor element, and wherein the first flow path rectilinearly connects the side of the inner wall and the side of the outer wall to each other in the NULL area in the direction orthogonal to the center axis direction of the first housing.

(5) The electronic stethoscope according to any one of (1) to (4), wherein the first housing includes a first wall portion that defines, together with the shielding member, a first space located closer to the first end than the sensor element, and a second wall portion that defines a second space located closer to the second end than the sensor element, and that includes a cylindrical outer wall and a tapered inner wall having a diameter decreasing in a direction toward the sensor element, and wherein the second space is defined by the inner wall of the second wall portion and the sensor element.

(6) The electronic stethoscope according to (5), wherein the first flow path connects the first space and an outside of the first housing to each other.

(7) The electronic stethoscope according to (5), wherein the first flow path connects the side of the inner wall of the second wall portion and the side of the outer wall of the second wall portion to each other.

(8) The electronic stethoscope according to any one of (5) to (7), further including: a substrate disposed between the first wall portion and the second wall portion and connected to the sensor element; and a venting member disposed in the NULL area, separating the first wall portion and the second wall portion from each other together with the substrate, and allowing air to pass therethrough.

(9) The electronic stethoscope according to any one of (5) to (8), further including: a second housing disposed closer to the first end of the first housing, and including a third wall portion that defines a third space adjacent to the first space, wherein the first flow path connects the first space and the third space to each other, and wherein the second housing includes a second flow path that connects the third space and an outside of the electronic stethoscope to each other.

(10) The electronic stethoscope according to (9), wherein the second flow path is disposed in the NULL area, and extends in a direction crossing the first flow path.

(11) The electronic stethoscope according to (9), wherein the second flow path is disposed outside the NULL area.

(12) The electronic stethoscope according to any one of (9) to (11), further comprising: a sound absorber disposed in the third space.

(13) The electronic stethoscope according to any one of (1) to (12), wherein the sensor element is formed from an electret condenser microphone, a piezoelectric vibration plate, or a cylindrical piezoelectric microphone.

(14) The electronic stethoscope according to any one of (1) to (13), further comprising: a diaphragm disposed at the second end of the first housing.

(15) The electronic stethoscope according to any one of (1) to (14), wherein the NULL area is an area where sensitivity of the sensor element is reduced by greater than or equal to 10 dB with respect to a maximum gain.

(16) An electronic stethoscope that captures biometric sound, including: a sensor element that captures biometric sound; a substrate on which the sensor element is disposed; and a first housing that accommodates the sensor element, that has a pipe shape having a first end and a second end, and that receives a shielding member at the first end, wherein the first housing includes a first flow path that extends through the first housing and that connects a side of an inner wall and a side of an outer wall of the first housing to each other, and wherein the first flow path is located at a position not overlapped with the sensor element and the substrate when viewed in a vertical direction and a horizontal direction from the sensor element.

The present disclosure is applicable to an electronic stethoscope for capturing biometric sound.

What is claimed is:

1. An electronic stethoscope configured to capture biometric sound, comprising:
a sensor element configured to capture biometric sound; and
a first housing accommodating the sensor element, the first housing having a pipe shape having a first end and a second end, the first housing receiving a shielding member at the first end,
wherein the first housing includes a first flow path extending through the first housing and connecting a side of an inner wall and a side of an outer wall of the first housing to each other,
wherein, around the sensor element, a NULL area in which sensitivity of the sensor element is reduced by greater than or equal to a predetermined value with respect to a maximum gain at which the sensitivity of the sensor element is maximum is defined, and
wherein at least one of an opening of the first flow path provided in the inner wall of the first housing or an opening of the first flow path provided in the outer wall of the first housing is located in the NULL area of the sensor element.

2. The electronic stethoscope according to claim 1,
wherein the first flow path rectilinearly connects the side of the inner wall and the side of the outer wall to each other in the NULL area in a direction toward the sensor element.

3. An electronic stethoscope configured to capture biometric sound, comprising:
a sensor element configured to capture biometric sound; and
a first housing accommodating the sensor element, the first housing having a pipe shape having a first end and a second end, the first housing receiving a shielding member at the first end,
wherein the first housing includes a first flow path extending through the first housing and connecting a side of an inner wall and a side of an outer wall of the first housing to each other,
wherein, around the sensor element, a NULL area is defined by at least one of: extending from the sensor element toward the first end or extending in a direction orthogonal to a center axis direction of the first housing from the sensor element, and
wherein the first flow path rectilinearly connects the side of the inner wall and the side of the outer wall to each other in the NULL area in a center axis direction of the first housing.

4. The electronic stethoscope according to claim 1,
wherein the first housing includes
a first wall portion defining, together with the shielding member, a first space located closer to the first end than the sensor element, and
a second wall portion defining a second space located closer to the second end than the sensor element, the second wall portion including a cylindrical outer wall and a tapered inner wall having a diameter decreasing toward the sensor element, and
wherein the second space is defined by the inner wall of the second wall portion and the sensor element.

5. The electronic stethoscope according to claim 4,
wherein the first flow path connects the first space and an outside of the first housing to each other.

6. The electronic stethoscope according to claim 4,
wherein the first flow path connects the side of the inner wall of the second wall portion and the side of the outer wall of the second wall portion to each other.

7. The electronic stethoscope according to claim 4, further comprising:
a substrate disposed between the first wall portion and the second wall portion and connected to the sensor element; and
a venting member disposed in the NULL area, separating the first wall portion and the second wall portion from each other together with the substrate, and allowing air to pass therethrough.

8. The electronic stethoscope according to claim 4, further comprising:
a second housing disposed closer to the first end of the first housing, and including a third wall portion that defines a third space adjacent to the first space,
wherein the first flow path connects the first space and the third space to each other, and
wherein the second housing includes a second flow path that connects the third space and an outside of the electronic stethoscope to each other.

9. The electronic stethoscope according to claim 8,
wherein the second flow path is disposed in the NULL area and extends in a direction crossing the first flow path.

10. The electronic stethoscope according to claim 8,
wherein the second flow path is disposed outside the NULL area.

11. The electronic stethoscope according to claim 8, further comprising:
a sound absorber disposed in the third space.

12. The electronic stethoscope according to claim 1,
wherein the sensor element is provided from an electret condenser microphone, a piezoelectric vibration plate, or a cylindrical piezoelectric microphone.

13. The electronic stethoscope according to claim 1, further comprising:
a diaphragm disposed at the second end of the first housing.

14. The electronic stethoscope according to claim 1,
wherein the NULL area is an area where sensitivity of the sensor element is reduced by greater than or equal to 10 dB with respect to a maximum gain.

15. An electronic stethoscope configured to capture biometric sound, comprising:
a sensor element configured to capture biometric sound;
a substrate on which the sensor element is disposed; and
a first housing accommodating the sensor element, the first housing having a pipe shape having a first end and a second end, the first housing receiving a shielding member at the first end,
wherein the first housing includes a first flow path extending through the first housing and connecting a side of an inner wall and a side of an outer wall of the first housing to each other, and
wherein the first flow path is located at a position not overlapped with the sensor element and the substrate when viewed in a vertical direction and a horizontal direction with respect to the sensor element.

16. The electronic stethoscope according to claim 2,
wherein the first housing includes
a first wall portion defining, together with the shielding member, a first space located closer to the first end than the sensor element, and
a second wall portion defining a second space located closer to the second end than the sensor element, the second wall portion including a cylindrical outer wall and a tapered inner wall having a diameter decreasing toward the sensor element, and
wherein the second space is defined by the inner wall of the second wall portion and the sensor element.

17. The electronic stethoscope according to claim 3,
wherein the first housing includes
a first wall portion defining, together with the shielding member, a first space located closer to the first end than the sensor element, and
a second wall portion defining a second space located closer to the second end than the sensor element, the second wall portion including a cylindrical outer wall and a tapered inner wall having a diameter decreasing toward the sensor element, and
wherein the second space is defined by the inner wall of the second wall portion and the sensor element.

18. The electronic stethoscope according to claim 5, further comprising:
a substrate disposed between the first wall portion and the second wall portion and connected to the sensor element; and
a venting member disposed in the NULL area, separating the first wall portion and the second wall portion from each other together with the substrate, and allowing air to pass therethrough.
